(19) United States
(12) Patent Application Publication
(10) Pub. No.: US 2009/0202040 A1
(43) Pub. Date: Aug. 13, 2009

(54) Apparatus for the Production of Electron Beams and X-Ray Beams for Interstitial and Intra-Operatory Radiation Therapy

(76) Inventors: Marco Sumini, Alessandria (IT); Agostino Tartari, Mirandola (IT); Domiziano Mostacci, San Benedetto Val Di Sambro (IT)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(21) Appl. No.: 11/989,610

(22) PCT Filed: Aug. 3, 2006
(86) PCT No.: PCT/EP2006/007686

§ 371 Date: Feb. 23, 2009

(30) Foreign Application Priority Data
Aug. 4, 2005 (IT) VE2005A000037

(51) Int. Cl. A61N 5/10; A61N 5/00

(52) U.S. Cl. 378/65; 250/492.3

(57) ABSTRACT

Apparatus for the production of electron beams and of X-ray beams for interstitial and intra-operatory radiation therapy, characterized in that it comprises:—as a pulsed source of electrons, a head (2) of the Plasma Focus kind, with a vacuum chamber (4) provided with a pair of cylindrical, coaxial electrodes (6, 8) to generate an electrical discharge, and with means to introduce at least one reaction gas into the chamber (4)—an electrical circuitry feeding said source head, comprising a capacitor bank (12) with fast switches (14) and conductors (16, 21) connecting to the electrodes (6, 8) of said vacuum chamber (4), and—an electron guide (22), coaxial to said electrodes, departing from said source head to right near the irradiation site—an armored gantry (26, 28) with high voltage cables and means for the suspension and movement of the head (2).

The present invention refers to an apparatus for the production of electron beams and X-ray beams for interstitial and intra-operatory radiation therapy, more commonly named IORT (Intre Operative Radio Therapy). More specifically, the invention refers to an apparatus IORT using a Plasma Focus type of machine, coupled to an electron guide which conveys the beam directly to the tissue to be treated with a low-energy, extremely high dose rate of electron or photonic X-ray irradiation.

Plasma Focus machines of the Mather type (J W Mather, in: Methods of experimental physics, Vol. 9, Part B. Plasma Physics, Eds. RH Lovberg and HR Griem—Academic Press, New York, 1971—chapter 55) are known to be capable of generating, accelerating and confining plasma, which, under appropriate operating conditions, gives rise to thermonuclear reactions producing radiation and nuclides and/or other sub-atomic particles.

This kind of machine, particular in characteristics and performances, is described in the Italian Patent Application VE2004A000038 filed on Oct. 21, 2004 in the names of Marco SUMINI, Agostino TARTARI, Domiziano MOSTACCI, concerning an apparatus for endogenous production of radioisotopes, particularly for Positive Electron Tomography.

The technical field to which the apparatus of this invention is destined to is the post-surgical radiation therapy of tissue, following to the removal of small tumor masses. The apparatus is of particular interest to IORT in the four “big killers” of mankind, i.e. tumors of the lung, breast, prostate and low-intestine. The latter is the first cause of death due to malignant neoplasia amongst the Italian males.

Some of the IORT techniques presently used release the required dose to the site by means of beams of electrons or photons (gamma or X), either in continuous or pulsed mode.

As an example, the Photon Radiosurgery System (PRS) by Photoelectron Corporation of Massachusetts (USA) is known. It is a miniature accelerator of electrons, of energy in the order of 40÷50 keV, that are focused through a 10 cm long, 3.2 mm diameter guide on a thin target to produce a continuous beam of X-rays.

Another known apparatus giving similar performances is the Intrabeam™, by Oncology Systems Limited, Battlefield—United Kingdom in partnership with Carl ZEISS.

Yet another known example is the movable linear accelerator Novac7 produced by Hytesis of Latina (Italy): this generates a linear beam of electrons of energy between 3 and 9 MeV. The beam is guided by a Plexiglas applicator measuring several centimeters of diameter.

In these known apparatuses the amount of dose released does not wholly determine the radiobiological damage, as the latter is strictly related both to time and space conditions of the release. For this reason, together with the traditional continuos therapy planning, techniques have recently been developed which deliver the dose fractionally (in time and space), by means or radioisotope implants. Use of beams of particles with high therapeutic efficiency (i.e. neutrons, protons and ions) has been introduced in medical practice, albeit for specific cases. Their production, however, requires huge plants like nuclear reactors, synchrotrons or large linear accelerators.

Apart from the requirement of high radiobiological efficiency, IORT has at least three mandatory requirements:
treatment in the vicinity of the surgery room
exact location of the dose released
minimum possible irradiation time and the possibility of concluding the treatment in a single IORT session

None of the previously mentioned apparatuses, nor others occasionally proposed, satisfy all the above requirements.

In the light of this state of the art, the main problem that the present invention intends to tackle and solve is that of finding an alternative to the existing IORT systems, overcoming their main drawbacks such as:
the difficulties in collimating the beam, and hence in treating small (few millimeters) tumors
the low dose rate transferred to the tissue under treatment
the low radiobiological effectiveness of the beams
the cumbersome size of the irradiation head
the difficulty in controlling and circumscribing the risks for the patient
the excessive length of time of each treatment
no choice of the type of radiation (either X-rays or electrons)

According to the present invention all these problems are solved with an apparatus for the production of electron beams and of X-ray beams for interstitial and intra-operatory radiation therapy, characterized in that it comprises:
as a pulsed source of electrons, a head of the Plasma Focus kind, with a vacuum chamber provided with a pair of cylindrical, coaxial electrodes to generate an electrical discharge, and with means to introduce at least one reaction gas into the chamber
an electrical circuitry feeding said source head, comprising a capacitor bank with fast switches and conductors connecting to the electrodes of said vacuum chamber, and
an electron guide, coaxial to said electrodes, departing from said source head right near to the irradiation site
an armored gantry with high voltage cables and means for the suspension and movement of the head

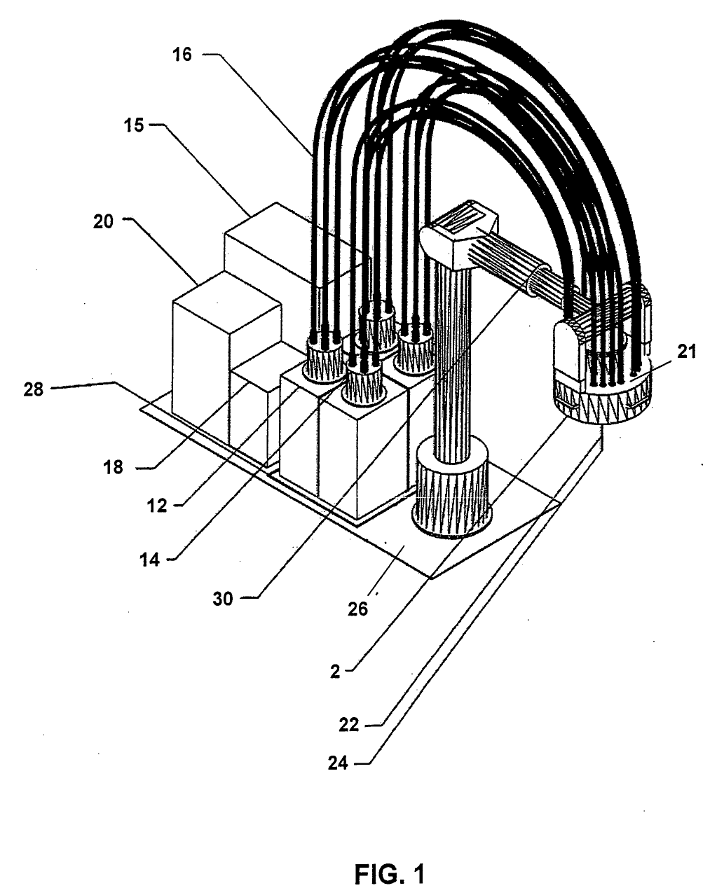

FIG. 1 shows a general, schematic perspective view of an apparatus according to the invention

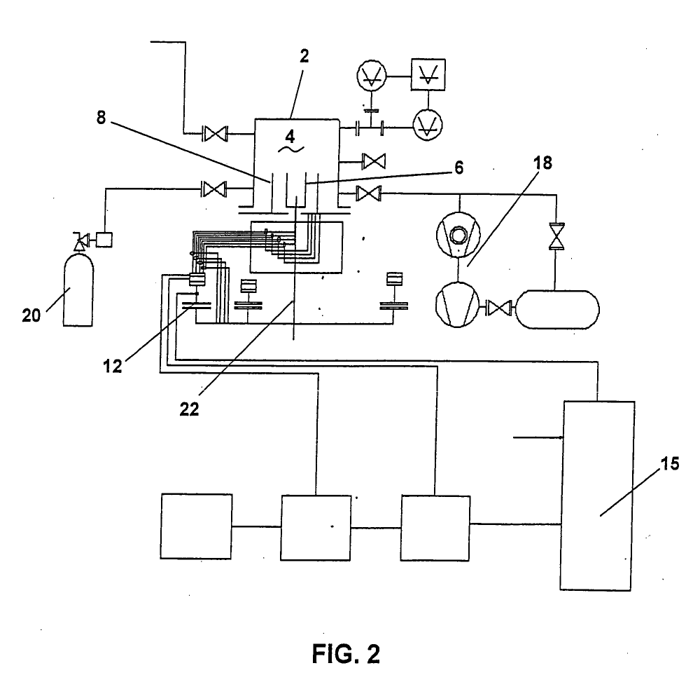

FIG. 2 shows its electrical and hydraulic scheme

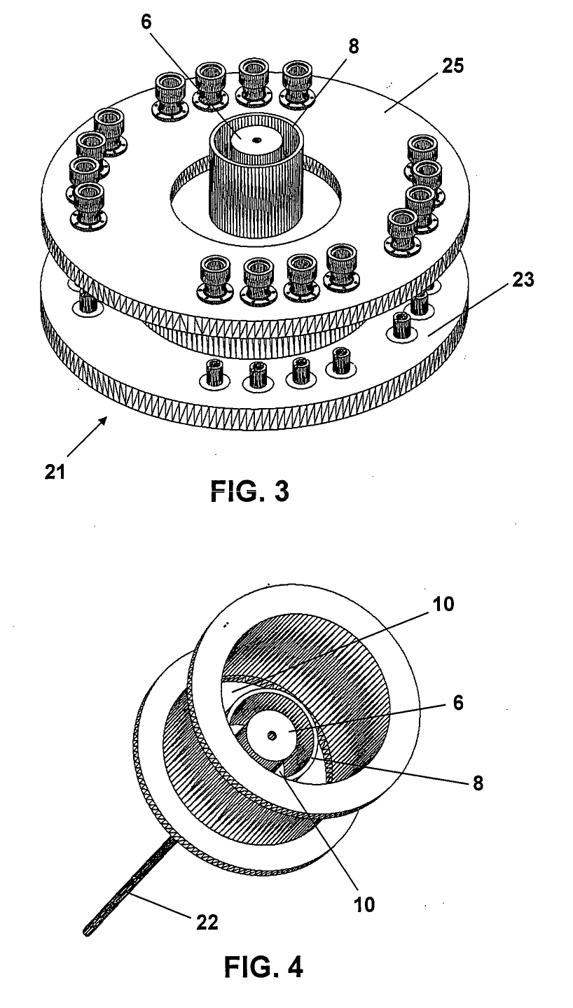

FIG. 3 shows the detail of the electrodes, the vacuum chamber, and the manifold

FIG. 4 shows an enlarged view of the electrodes of the vacuum chamber and of the electron guide

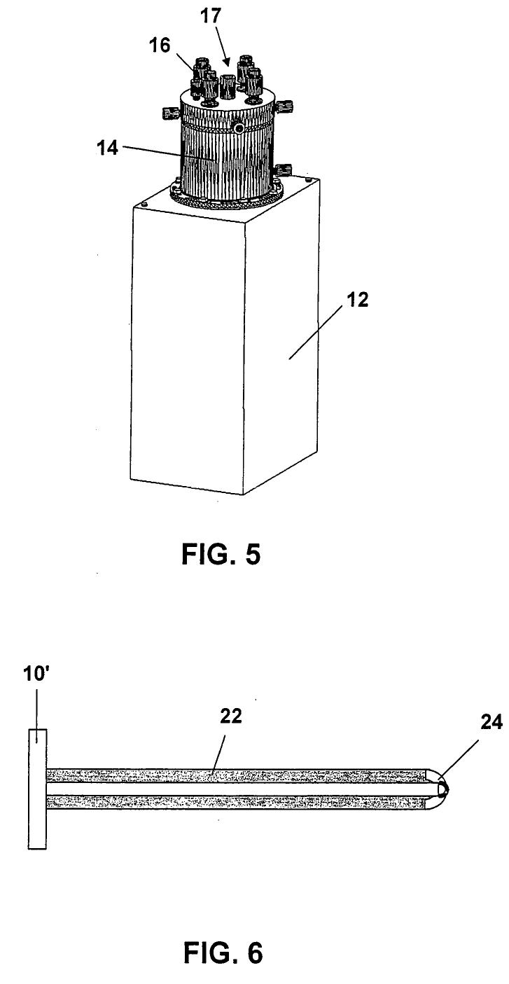

FIG. 5 shows a capacitor with its fast switch

FIG. 6 shows a schematic, longitudinal section of the electron guide

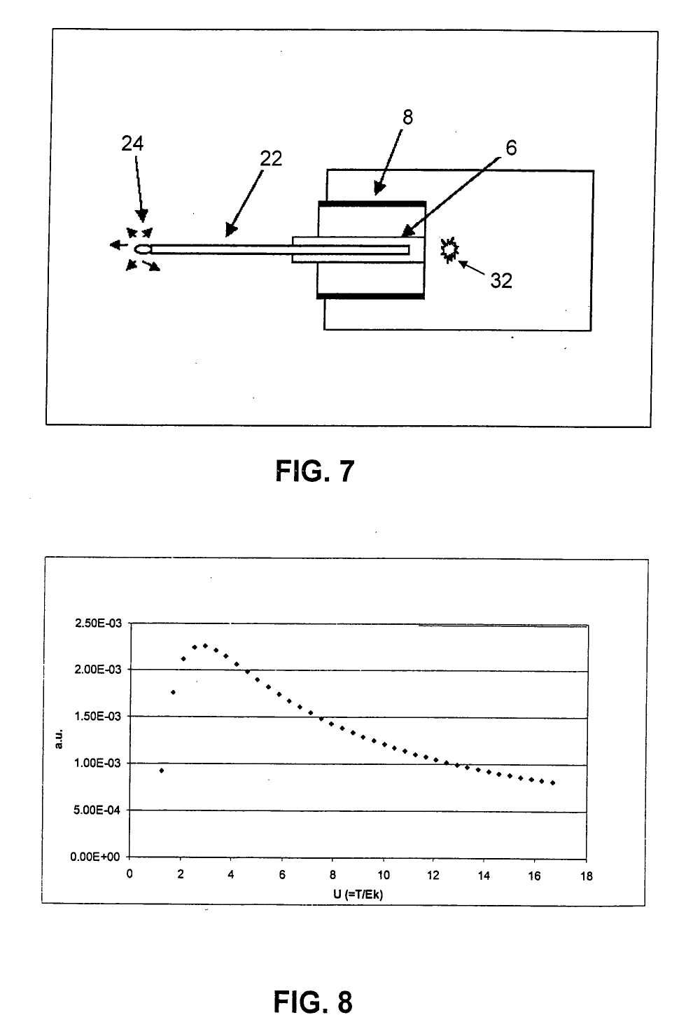

FIG. 7 shows the functional scheme of the vacuum chamber with the electrodes and the electron guide, and

FIG. 8 shows the pattern of the X-ray production as a function of the incident electron energy, in the case of a tungsten converter (experimental data)

As can be seen in figures, the apparatus according to the invention may be defined a machine for the production of beams of electrons and X-rays for IORT treatment. It comprises a source-head 2 of electron pulses, based on a special version of Mather-type Plasma Focus. More specifically, the source-head 2 comprises a vacuum chamber 4 enclosing two cylindrical coaxial electrodes 6,8 separated by an insulator 10, which also closes the bottom of the chamber 4.

The two electrodes 6,8 are connected to a capacitor bank 12 through fast switches 14. More specifically, the capacitors 12 are connected upstream to a charging power supply 15 and downstream through the fast switches 14 to a high voltage, high current transmission line 16 connecting to the electrodes 6,8. The fast switches 14 are connected on their side to a cooling circuit by means of proper connectors (not numbered in FIG. 5), and are connected on their upper part to the power supply 15 by means of a proper connector 17.

The vacuum chamber 4 is also equipped with connecting means to a vacuum pump 18 and with further connecting means to a Hydrogen, Neon or Argon supply 20.

The connection of the vacuum chamber 4 with the capacitor bank takes place directly in the bottom part of the chamber itself by means of particular manifolds 21 consisting in two discoidal steel disks, coaxial to each other, of which the lower disk 23 is connected to the inner electrode 6 of the vacuum chamber, and the upper disk 25 is connected to its outer electrode 8. The connection to the bank is effected by means of coaxial cables, the external conductor of which connects to the upper manifold disk 23 by a suitable connector, whereas the inner conductor crosses said upper manifold disk and ends in a suitable connector on the lower manifold disk 25. The connection between the manifold and the vacuum chamber takes place in the bottom of the chamber, where the two electrodes 6,8 are separated by the insulator. Here the upper manifold disk 25, receiving the external conductor of the coaxial cables, is connected to the external part of the vacuum chamber 4 and with the external electrode 8, while the lower manifold disk 23, receiving the central conductor of the coaxial cables, is connected to the lower plate of the central anode 6.

An electron guide is attached to the vacuum chamber 4. It is formed by a cylinder 22 hollow along its axis, of a material opaque to electrons.

For reasons of radiation protection, the material of the cylinder 22, in addition to fulfilling the mechanical strength and vacuum capability requirements, needs to yield the lowest possible production of X-rays from electron impact, both characteristic (X-K and X-L) and from “Bremsstrahlung”. Plexiglas meets fully all these criteria, and its physical and chemical properties are very well known, which makes designing easier. An alternative choice considered is stainless steel.

The electron guide is attached in a vacuum manner to the lower, insulating slab of the vacuum chamber 4, this latter possessing a central hole. At the opposite end, which is outside the vacuum chamber, the guide is fitted with an element 24 to vacuum-seal the guide 22 while permitting the extraction of the electrons.

Four different embodiments are foreseen for the fitting 24: a first embodiment consisting in a foil a few microns thick, in beryllium or other low-Z metal such that electrons can be extracted. A second embodiment consisting in a Mylar layer 10-12 micron thick, coated with a very thin layer of Aluminum, the Aluminum layer increasing the mechanical strength and thermal conductivity for the cooling of the Mylar layer. A third embodiment consisting in using layers 10-15 micron thick in Titanium, Tantalum or Tungsten. A fourth embodiment consisting in a half sphere of metal a few microns thick, to convert electron energy into characteristic X-rays, produced from ionization of the atoms in the foil from electron impact (A. Tartari et al: Energy spectra measurements of X-ray emission from electron interaction in a dense plasma focus devices, Nucl. Instr. Meth. B 213 (2004) 206).

In this fourth embodiment of the vacuum-tight fitting 24 the material of the foil is to be chosen to optimize characteristic X-ray production, considering the energy of the incident electrons. Yield as a function of energy shows that maximum production is obtained for incident electron energy three times larger than binding energy E_{K} of the K or L electrons: this can be seen in FIG. 8 for the case of tungsten as converter material.

In practical usage of the apparatus according to the invention for IORT treatment, the former is supported by a gantry comprising a wheeled base 26, in which the capacitor bank 12 is contained, wrapped in a Faraday's cage 28 to insulate electromagnetic fields. In FIG. 1, for clarity, the cage is not reported: however its base perimeter is indicated. On each capacitor 12 a corresponding fast switch 14 is fitted.

On the base are also attached articulated arms 30 supporting the vacuum chamber 4 hovering above the patient. The electron guide 22, possibly equipped with the converter to generate X-ray energy from electron energy, departing from the vacuum chamber then reaches the immediate vicinity of the site to be irradiated.

The operation of the apparatus according to the invention will now be described, for simplicity, with reference to a single pulse, albeit repetitive operation, at a 1 Hz frequency, is expected to be more appropriate for IORT.

In principle, the operation is as follows: the capacitor bank is charged to a specified voltage, then the energy stored is discharged very rapidly (few microseconds) into the electrodes 6,8. The discharge of the capacitors produces ionization of the gas between the electrodes, its transition to the state of plasma, and its motion toward the end of the electrodes. More specifically, the plasma sheet thus generated is pushed along the electrodes by the self-generated electromagnetic force, and accelerated toward the open end of the electrodes. Once the end is reached, the plasma is compressed by the intense electromagnetic fields generated, and implodes, producing the confinement conditions typical of thermonuclear plasmas, corresponding to a combination of density, energy and confinement time of the plasma particles of the order of 10¹⁵ keV·sec/cm³.

Said confinement takes place in a cylinder of approximately 1 mm diameter and 1 cm length, called pinch or focus (32, see FIG. 7). The lifetime of the focus, depending on bank energy, varies from 10 ns (6-7 kJ) to a few tens of nanoseconds (higher bank energies); at the end it decays, producing beams of particles.

When the filling gas is Hydrogen or Argon or Neon, in the focus there is production of:
beams of low energy X-rays and protons, emitted axially forward
beams or relativistic electrons (Relativistic Electron Beam—REB) with energy <1 MeV, emitted backward (electron fluency ˜0.5-5 mC/pulse for a 6-7 kJ machine).

Whereas X-ray and proton beams are easily absorbed in the walls of the vacuum chamber 4, the particular coaxial geometry of the electrodes 6,8 and electron guide 22, together with the hollow construction of the central electrode 6, permit extraction of the relativistic electron beams REB.

The electrons travel along the guide 22 and at its end are made available for IORT treatment, either directly or after conversion into 25-50 keV X-rays, according to needs and to the fitting 24 used.

From what was said above, it results clearly that the apparatus according to the invention is particularly valuable in comparison to known IORT systems, and in particular it presents the following advantages:
a high radiobiological effectiveness of the beams produced
limited encumbrance, considering that the source-head may have dimensions of about 20 cm×30 cm
dose release in well circumscribed and controlled sites, involving an area smaller than one square centimeter and a depth of 2-3 mm.
short overall treatment duration, estimated of the order of a minute
capability of using both electron and X-ray beam therapies

The following example, concerning a prototype already built and in an advanced stage of testing, will help clarify further the invention.

The IORT apparatus with Plasma Focus technology, in one specific embodiment, includes a Plasma Focus and is characterized by an electron guide made of a REB (Relativistic Electron Beam) measure chamber specifically designed to accommodate both the interchangeable X-ray converters and an X-ray spectrometer.

The parameter settings of the preliminary tests and the results obtained are the following:

Total capacitance of the condenser bank: 44.4 μF, each condenser having the following specifications:
Model: GA 32899
Capacitance: C=11.1 μF
Maximum Working Voltage: Vₘₐₓ=36 kV
Maximum Damage Voltage: V_{damage}=40 kV
Peak Working Current: I_{C}=150 kA
Working Voltage Reversal: 60%
Maximum Voltage Reversal: 80%
Life Cycles in Working Conditions: 1E6
Inductance: L_{C}=30 nH
Dimensions: 31×41×68 cm
Weight: 140 Kg

### Working Voltage: 20-26 KV.

### Bank Energy: 10-15 KJ.

### Total Inductance: 100 nH.

### Electrode Length: 13.3 cm.

Inner Electrode Diameter (copper): 3 cm.
Outer Electrode Diameter (copper): 8 cm.

### Pseudoperiod τ: 10-12 μs.

Volume of the Stainless Steel Vacuum Chamber: 2.5 dm³.
The power supply has the following specifications:
Output Voltage: V₀=20-30 kV
Deliverable Energy: 10-15 kJ
Bank Charging Time: 0.5÷0.8 s.
Average Current Delivered: I_{PS}=2 A
Average Power Delivered: P_{PS}=I_{PS}V_{O}=60 kVA.

The power supply can be driven both in single pulse mode and in repetitive mode: the latter making use of the timing of the fast switch trigger unit.

The fast switches have the following specifications:
Model: REB3 SG-182 o SG-183 Montecuccolino Type (i.e. specifically designed by R.E. Beverly III & Ass. according to specifications)
Triggering Type: field distortion
Minimum Working Voltage: 15 kV
Maximum Working Voltage: 65 kV
Working Current Peak: I_{SG}=160 kA
Maximum Current Peak: 250 kA
Maximum Transferable Charge Per Pulse: 0.36 C
Inductance: L_{SG}=27 nH
Closing Time: 22 ns
Breakdown Time: 600 ns
Working Gas: synthetic air
Output to Electrodes: 4 coaxial cables per switch
   Coaxial cables: DS 2248 by Dielectric Science.

The apparatus relies on a system for the cooling of the electrodes, on a system for the recirculation of the gas in the vacuum-chamber and a system for the recirculation of the fast switch working gas, always for cooling purposes, which have permitted the working in repetitive mode of the apparatus at a frequency of 1 Hz or greater.

The inductance of a single capacitor-fast switch unit is L_{C}+L_{SG}=57 nH

Capacitors were paralleled during charge, a distribution box was adopted containing high voltage diodes and appropriate circuitry for the suppression (“snubbing”) of disturbance.

The coaxial cables (16) connecting the four capacitor-fast switch units to the manifold, in turn connected to the electrodes (6,8), are 4 per unit, i.e. a total of 16, each carrying a maximum current ¼ I_{Cmax}≅25 kA.

Each coaxial cable is 4.2 m long.

The manifold disks (21) have diameter 45 cm.

Tests have shown that the X-L spectral component of characteristic X-rays accounts for 35% of the spectrum and their energy is 8.9 keV, while the “Bremsstrahlung” component accounts for the remaining 65%, with an energy of 25.0 keV and the dose delivered in a shot is 10 Gy in a time span of 30 ns.

1. An apparatus for the production of electron beams and of X-ray beams for interstitial and intra-operatory radiation therapy, comprising
a source head, with a vacuum chamber provided with a pair of cylindrical, coaxial electrodes having an external electrode and an internal electrode to generate an electrical discharge, and with means to introduce at least one reaction gas into the chamber
an electrical circuitry feeding said source head, comprising a capacitor bank with fast switches and conductors connecting to the electrodes of said vacuum chamber, and
an electron guide, coaxial to said electrodes, departing from said source head to near the irradiation site
an armored gantry with high voltage cables and means for the suspension and movement of the head.

2. The apparatus according to claim 1 wherein the source head is of the Mather type.

3. The apparatus according to claim 1 wherein the source head comprises a vacuum chamber, in which the two coaxial electrodes are separated by an insulator.

4. The apparatus according to claim 1 wherein means of electrical connection of the fast switches to the electrodes of the vacuum chamber is at least one coaxial cable.

5. The apparatus according to claim 1 further comprising a manifold comprising two coaxial disks electrically connected to the two coaxial electrodes of the source head, the external conductor of the coaxial cables being connected to the upper disk of the manifold, and the central conductor of the cables being connected to the lower disk after having crossed said upper outer disk.

6. The apparatus according to claim 4 wherein the external electrode in the vacuum chamber is connected to ground and to the external conductor of at least one coaxial cable, while the internal electrode is connected to the inner conductor of at least one coaxial cable.

7. The apparatus according to claim 1 wherein the electron guide comprises a cylinder, hollow along its axis, made in a material opaque to electrons and connected vacuum-tightly to the lower slab of the source head in correspondence to a central hole of the latter; said guide having fitted at the opposite end a fitting to vacuum-seal the guide.

8. The apparatus according to claim 1 wherein the electron guide is built with a material having low efficiency in the production of X-rays from electron impact.

9. The apparatus according to claim 8 wherein the electron guide is polymethyl methacrylate.

10. The apparatus according to claim 8 wherein the electron guide is stainless steel.

11. The apparatus according to claim 7 wherein the fitting comprising a thin foil of a metal having low atomic number.

12. The apparatus according to claim 11 wherein the fitting is beryllium.

13. The apparatus according to claim 11 wherein the fitting is titanium.

14. The apparatus according to claim 11 wherein the fitting is tantalum.

15. The apparatus according to claim 11 wherein the fitting is biaxially-oriented polyethylene terephthalate coated with aluminium.

16. The apparatus according to claim 7 wherein the fitting comprises a thin foil of a material fit for converting the energy of impinging electrons into characteristic X-rays, produced from ionization of atoms following electron impact.

17. The apparatus according to claim 16 wherein the fitting is built with a material having binding energy E_{K} of K and L electrons that is approximately ⅓ of the energy of impinging electrons.

18. The apparatus according to claim 17 wherein the fitting is tungsten.

19. The apparatus according to claim 1 wherein the gantry includes a base containing the capacitor bank and the fast switches.
